(19) United States
(12) Patent Application Publication
(10) Pub. No.: US 2002/0142369 A1
(43) Pub. Date: Oct. 3, 2002

(54) Refolding method of thrombin

(76) Inventor: Alan Roy Fersht, Cambridge (GB)

Correspondence Address: FROMMER LAWRENCE & HAUG LP, 745 Fifth Avenue, New York, NY, 10151, US

(21) Appl. No.: 10/033,737

(22) Filed: Nov. 13, 2001

Related U.S. Application Data
(63) Continuation-in-part of application No. PCT/GB00/01827, filed on May 12, 2000.

(30) Foreign Application Priority Data
May 19, 1999 (GB) 9911689.9

(51) Int. Cl. C12P 21/06

(52) U.S. Cl. 435/68.1

(57) ABSTRACT

A method for promoting the foling of a polypeptide selected from thrombin and a precursor thereof comprising contacting the polypeptide with a molecular chaperone and a foldase is provided.

### FIELD OF THE INVENTION

The present invention relates to a method for refolding recombinant prethrombin and thrombin.

### BACKGROUND TO THE INVENTION

Thrombin is a multifunctional protease playing a key role in the blood-clotting cascade. It has a very high specificity and is used in the laboratory as a reagent to cleave at specific sites in a protein. The specificity sequence is often inserted into recombinant proteins, between their functional regions and a synthetic linker sequence that is designed to attach them to other proteins, and also to amino acid sequences that can be selectively attached to chromatography columns. Cleavage by thrombin is used to release the desired protein. Thrombin is thus a very important reagent for protein purification.

Thrombin can be isolated directly from mammals in small amounts. Existing production of recombinant thrombin relies on expression in mammalian cell systems which all produce ˜0.5 to 8 μg of thrombin per ml of cell culture. Alternatively thrombin has to be purified from blood plasma which has the disadvantage of contamination by other clotting agents. Thrombin is thus a very expensive reagent. Recombinant thrombin expressed in Escherichia coli (E. coli) should be produced much more cheaply and have the important advantage of being more acceptable for use in biotechnology for the production of proteins because there would not be the possibility of contamination by mammalian proteins. DiBella et al. (1995) have produced unglycosylated bovine prethrombin-2 from an E. coli system which, when activated to thrombin with snake venom, has essentially the same catalytic activity as wild-type thrombin. However, they are only able to recover ˜1% active material from prethrombin-2.

Thus there is a need in the art for an improved method for producing recombinant thrombin in large quantities whilst maintaining high levels of activity.

### SUMMARY OF THE INVENTION

Using oxidative refolding chromatography, as previously described by Altamirano et al. (1999), we have shown that it is possible to increase the recovery of active protein from partly purified inclusion bodies to ˜15%, without further purification.

Accordingly the present invention provides a method for promoting the folding of a polypeptide selected from thrombin and a precursor thereof comprising contacting the polypeptide with a molecular chaperone and at least one foldase. It is especially preferred that the contacting takes place under reducing conditions.

Preferably the molecular chaperone and/or foldase(s) are immobilised to a solid phase, more preferably both the chaperone and foldase(s) are immobilised to a solid phase. Preferably the solid phase is a matrix. More preferably the matrix is present in a chromatography column.

Preferably the molecular chaperone is an hsp60 chaperonin or fragment thereof having refolding activity, more preferably a molecular chaperone fragment comprising a region consisting of fragments 191-376, 191-345 or 191-335 of the sequence of E. Coli GroEL or a homologue thereof.

Preferably the foldase is selected from a thiol/disulphide oxidoreductase and a peptidyl-prolyl isomerase. Preferably the thiol/disulphide oxidoreductase is selected from E. coli DsbA and mammalian protein disulphide isomerase and the peptidyl prolyl isomerase is independently selected from cyclophilin, parbulen, SurA and FK506 binding proteins. Preferably the PPI is cyclophilin A, advantageously mammalian, such as human cyclophilin A.

In a preferred embodiment the method of the invention comprises contacting the polypeptide with a molecular chaperone and both a thiol/disulphide oxidoreductase and a peptidyl-prolyl isomerase. Preferably, the thiol/disulphide oxidoreductase is DsbA and the peptidyl-prolyl isomerase is cyclophilin A.

The present invention also provides the use of a molecular chaperone and one or more foldases for promoting the folding of a polypeptide selected from thrombin and a precursor thereof.

In another aspect the invention provides a polypeptide selected from thrombin and a precursor thereof obtainable by the method of the invention. Said polypeptide is typically obtained at higher yields and having a higher specific activity than a thrombin polypeptide obtained using normal methods of protein expression in non-mammalian expression systems such as E. coli. A polypeptide of the invention may be used in protein purification. In particular said polypeptide may be used to cleave a heterologous polypeptide, preferably a heterologous polypeptide that has been produced recombinantly.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Current Protocols in Molecular Biology (1995), John Wiley & Sons, Inc.

A. Molecular Chaperones and Foldases

Molecular Chaperones

Chaperones, including chaperoning, are polypeptides which promote protein folding by non-enzymatic means, in that they do not catalyse the chemical modification of any structures in folding polypeptides, but promote the correct folding of polypeptides by facilitating correct structural alignment thereof. Molecular chaperones are well known in the art, several families thereof being characterised. The invention is applicable to any molecular chaperone molecule, which term includes, for example, the molecular chaperones selected from the following non-exhaustive group:

p90 Calnexin, HSP family, HSP70 family, DNA K, DNAJ, HSP60 family (GroEL) ER-associated chaperones, HSP90, Hsc70, sHsps; SecA; SecB, Trigger factor, zebrafish hsp 47, 70 and 90, HSP 47, GRP 94, Cpn 10, BiP, GRP 78, C1p, FtsH, Ig invariant chain, mitochondrial hsp70, EBP, mitochondrial m-AAA, Yeast Ydj 1, Hsp104, ApoE, Syc, Hip, TriC family, CCT, PapD and calmodulin (see W099/05163 for references).

Two major families of protein folding chaperones which have been identified, the heat shock protein 60 (hsp60) class and the heat shock protein 70 (hsp70) class, are especially preferred for use herein. Chaperones of the hsp60 class are structurally distinct from chaperones of the hsp70 class. In particular, hsp60 chaperones appear to form a stable scaffold of two heptamer rings stacked one atop another which interacts with partially folded elements of secondary structure. On the other hand, hsp70 chaperones are monomers of dimers and appear to interact with short extended regions of a polypeptide.

Hsp70 chaperones are well conserved in sequence and function. Analogues of hsp70 include the eukaryotic hsp70 homologue originally identified as the IgG heavy chain binding protein (BiP). BiP is located in all eukaryotic cells within the lumen of the endoplasmic reticulum (ER). The prokaryotic DnaK hsp70 protein chaperone in Escherichia coli shares about 50% sequence homology with an hsp70 KAR2 chaperone in yeast. Moreover, the presence of mouse BiP in yeast can functionally replace a lost yeast KAR2 gene.

Hsp60 chaperones are universally conserved and include hsp60 homologues from a large number of species, including man. They include, for example, the E coli GroEL polypeptide; Ehrlichia sennetsu GroEL; Trichomonas vaginalis hsp60; rat hsp60; and yeast hsp60.

In a preferred aspect, the present invention relates to fragments of polypeptides of the hsp60 family. These proteins being universally conserved, any member of the family may be used; however, in a particularly advantageous embodiment, fragments of GroEL, such as E. coli GroEL, are employed, especially those fragments termed minichaperones which are substantially monomeric in solution (see WO98/13496). Particularly preferred fragments of E. coli GroEL described in WO98/13496 are discussed below.

Chaperone activity may be determined in practice by an ability to refold cyclophilin A but other suitable proteins such as glucosamine-6-phosphate deaminase or a mutant form of indoleglycerol phosphate synthase (IGPS) (amino acid residues 49-252) may be used. A rhodanese refolding assay may also be used. Details of a suitable refolding assay are given below.

Preferred chaperone polypeptides of the present invention have protein refolding activity in the absence of adenosine triphosphate of at least 10%, advantageously 15%, preferably 25% and optimally more than 50%, preferably 60%, even more preferably 75%, said refolding activity being determined by contacting the chaperone polypeptide with an inactivated protein of known specific activity prior to inactivation, and then determining the specific activity of the said protein after contact with the polypeptide, the % refolding activity being:

$$\frac{specific activity of protein after contact with polypeptide}{specific activity of protein prior to inactivation } \times \frac{100}{1}$$

Preferably, the chaperone activity is determined by the refolding of cyclophilin A. More preferably, 8 M urea denatured cyclophilin A (100 μM) is diluted into 100 mM potassium phosphate buffer pH 7.0, 10 mM DTT to a final concentration of 1 μM and then contacted with at least 1 μM of said polypeptide at 25° C. for at least 5 minutes, the resultant cyclophilin A activity being assayed by the method of Fischer et al. (1984).

It is preferred that chaperone polypeptides of the present invention are monomeric in solution and incapable of multimerisation in solution. Monomeric GroEL minichaperones are disclosed in WO98/13496. Typically, multimerisation is prevented by using chaperone polypeptides that lack the interacting domains found outside the apical domain, although it could be achieved by suitable mutations.

The chaperone may be a circular permutation of a chaperone fragment sequence. Circular permutation is described in Graf and Schachman, PNAS(USA) 1996, 93:11591; this strategy is general for most of proteins whose N and C termini are closely spaced in tertiary structure. Circular permutated proteins keep their activity. Essentially, the polypeptide is circularised by fusion of the existing N and C termini, and cleavage of the polypeptide chain elsewhere to create novel N and C termini.

Foldases

In general terms, a foldase is an enzyme which participates in the promotion of protein folding through its enzymatic activity to catalyse the rearrangement or isomerisation of bonds in the folding polypeptide. They are thus distinct from a molecular chaperone, which bind to polypeptides in unstable or non-native structural states and promote correct folding without enzymatic catalysis of bond rearrangement. Many classes of foldase are known, and they are common to animals, plants and bacteria. They include peptidyl prolyl isomerases and thiol/disulphide oxidoreductases. The invention comprises the use of all foldases which are capable of promoting protein folding through covalent bond rearrangement.

Moreover, as used herein, the term “a foldase” includes one or more foldases. In general, in the present specification the use of the singular does not preclude the presence of a plurality of the entities referred to, unless the context specifically requires otherwise.

Thiol/Disulphide Oxidoreductase.

As the name implies, thiol/disulphide oxidoreductases catalyse the formation of disulphide bonds and can thus dictate the folding rate of disulphide-containing polypeptides. The invention accordingly comprises the use of any polypeptide possessing such an activity. This includes chaperone polypeptides, or fragments thereof, which may possess protein disulphide isomerase activity. In eukaryotes, thiol/disulphide oxidoreductases are generally referred to as protein disulphide isomerases (PDIs). PDI interacts directly with newly synthesised secretory proteins and is required for the folding of nascent polypeptides in the endoplasmic reticulum (ER) of eukaryotic cells.

Enzymes found in the ER with PDI activity include mammalian PDI, yeast PDI, mammalian ERp59, mammalian prolyl4-hydroxylase, yeast GSBP and mammalian T3BP, A. niger PdiA and yeast EUGI (see WO99/05163 for references). In prokaryotes. equivalent proteins exist, such as the DsbA protein of E. coli. Other peptides with similar activity include, for example, p52 from T cruzi. These polypeptides, and other functionally equivalent polypeptides, are included within the scope of the present invention, as are derivatives of the polypeptides which share the relevant activity (see below). Preferably, the thiol/disulphide oxidoreductase according to the invention is selected from mammalian PDI or E. coli DsbA.

Peptidy-Prolyl Isomerase.

Peptidyl-prolyl isomerases (PPIs) are present in a wide variety of cells. Known examples include cyclophilin, parbulen, SurA and FK506 binding proteins FKBP51 and FKBP52 (see WO99/05163 for references). PPI is responsible for the cis-trans isomerisation of peptidyl-prolyl bonds in polypeptides, thus promoting correct folding. The invention includes any polypeptide having PPI activity. This includes chaperone polypeptides, or fragments thereof, which may possess PPI activity.

Derivatives, Variants and Fragments.

The present invention relates to derivatives of molecular chaperones and foldases (such as peptidyl-prolyl isomerases and thiol/disulphide oxidoreductases). In a preferred aspect, therefore, the terms “molecular chaperone”, “peptidyl-prolyl isomerase” and “thiol-disulphide oxidoreductase” include derivatives thereof which retain the stated activity. The derivatives which may be used according to the present invention include splice variants encoded by mRNA generated by alternative splicing of a primary transcript, amino acid mutants, glycosylation variants and other covalent derivatives of molecular chaperones or foldases which retain the functional properties of molecular chaperones, peptidyl-prolyl isomerases and/or thiol/disulphide oxidoreductases.

Exemplary derivatives include molecules which are covalently modified by substitution, chemical, enzymatic, or other appropriate means with a moiety other than a naturally occurring amino acid. Such a moiety may be a detectable moiety such as an enzyme or a radioisotope. Further included are naturally occurring variants of molecular chaperones or foldases found within a particular species, whether mammalian, other vertebrate, yeast, prokaryotic or otherwise. Such a variant may be encoded by a related gene of the same gene family, by an allelic variant of a particular gene, or represent an alternative splicing variant of a molecular chaperone or foldase.

As noted above, the components of the combination according to the invention may comprise derivatives of molecular chaperones or foldases, including variants of such polypeptides which retain common structural features thereof. Variants which retain common structural features can be fragments of molecular chaperones or foldases. Fragments of molecular chaperones or foldases comprise smaller polypeptides derived from therefrom. Preferably, smaller polypeptides derived from the molecular chaperones or foldases according to the invention define a single feature which is characteristic of the molecular chaperones or foldases. Fragments may in theory be almost any size, as long as they retain the activity of the molecular chaperones or foldases described herein.

When applied to chaperone molecules, a fragment is anything other that the entire native molecular chaperone molecule which nevertheless retains chaperonin activity. Advantageously, a fragment of a chaperonin molecule remains monomeric in solution. Preferred fragments are described below. Advantageously, chaperone fragments are between 50 and 200 amino acids in length, preferably between 100 and 200 amino acids in length and most preferably about 150 amino acids in length.

With respect to molecular chaperones of the GroEL/hsp-60 family, a preferred set of fragments have been identified which possess the desired activity. These fragments are set forth in our copending international patent application WO98/13496 and in essence comprise any fragment comprising at least amino acid residues 230-271 of intact GroEL, or their equivalent in another hsp60 chaperone. Preferably, the fragments should not extend beyond residues 150-455 or 151-456 of GroEL or their equivalent in another hsp60 chaperones.

Advantageously, the fragments comprise the apical domain of GroEL, or its equivalent in other molecular chaperones, or a region homologous thereto as defined herein. The apical domain spans amino acids 191-376 of intact GroEL. This domain is found to be homologous amongst a wide number of species and chaperone types. In a highly preferred embodiment, the fragments are selected from fragments consisting essentially of residues 191-376, 191-345, 191-335 or 193-335 of the sequence of intact GroEL.

Derivatives of the molecular chaperones or foldases also comprise mutants thereof. including mutants of fragments and other derivatives, which may contain amino acid deletions, additions or substitutions, subject to the requirement to maintain the activity of the molecular chaperones or foldases described herein. Thus, conservative amino acid substitutions may be made substantially without altering the nature of the molecular chaperones or foldases, as may truncations from the 5′ or 3′ ends. Deletions and substitutions may moreover be made to the fragments of the molecular chaperones or foldases comprised by the invention. Mutants may be produced from a DNA encoding a molecular chaperone or foldase which has been subjected to in vitro mutagenesis resulting e.g. in an addition, exchange and/or deletion of one or more amino acids. For example, substitutional, deletional or insertional variants of molecular chaperones or foldases can be prepared by recombinant methods and screened for immuno-crossreactivity with the native forms of the relevant molecular chaperone or foldase.

The fragments, mutants and other derivative of the molecular chaperones or foldases preferably retain substantial homology with the native molecular chaperones or foldases. As used herein, “homology” means that the two entities share sufficient characteristics for the skilled person to determine that they are similar in origin and function. Preferably, homology is used to refer to sequence identity. Thus, the derivatives of molecular chaperones or foldases preferably retain substantial sequence identity with native forms of the relevant molecular chaperone or foldase.

“Substantial homology”, where homology indicates sequence identity, means more than 40% sequence identity, preferably more than 45% sequence identity and most preferably a sequence identity of at least 50%, 60% or more, as judged by direct sequence alignment and comparison.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al, 1984, Nucleic Acids Research 12:387). Examples of other software that can perform sequence comparisons include, but are not limited to, the BLAST package (see http://www.ncbi.nih.gov/BLAST/), FASTA (Atschul et al, 1990, J. Mol. Biol., 403410; FASTA is available for online searching at, for example, http://www.2.ebi.ac.uk.fasta3) and the GENEWORKS suite of comparison tools. However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The skilled person can identify suitable homologues by, for example, carrying out a search of online databases using all or part of a molecular chaperone/foldase sequence as a query sequence. For example, a search of the Swissprot database using the BlastP program Ver 2.0.8 (default settings) (Jinghui Zhang et al., 1997, Gapped BLAST and PSI-BLAST: a new generation of protein database search programs”, Nucleic Acids Res. 25:3389-3402) and amino acids 191 to 376 of E. coli GroEL as the query sequence identified well over a hundred homologous sequences, many of which gave homology scores of at least 50% identity. Homologues identified include members of the hsp60 chaperonin family which includes the eubacterial GroEL, mitochondrial hsp60 and chloroplast cpn60. Other specific homologues together with their database accession numbers are detailed in WO98/13496.

Alternatively, sequence similarity may be defined according to the ability to hybridise to a complementary strand of a nucleotide sequence encoding any of the chaperone or foldases mentioned above, such as E. coli GroEL, E. coli DsbA or mammalian cyclophilin A.

Preferably, the sequences are able to hybridise with high stringency. Stringency of hybridisation refers to conditions under which polynucleic acid hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na⁺ at 65-68° C. High stringency conditions can be provided, for example, by hybridisation in an aqueous solution containing 6× SSC, 5× Denhardt's, 1% SDS (sodium dodecyl sulphate), 0.1 Na⁺ pyrophosphate and 0.1 mg/ml denatured salmon sperm DNA as non specific competitor. Following hybridisation, high stringency washing may be done in several steps, with a final wash (about 30 minutes) at the hybridisation temperature in 0.2-0.1× SSC, 0.1% SDS.

Moderate stringency refers to conditions equivalent to hybridisation in the above described solution but at about 60-62° C. In that case the final wash is performed at the hybridisation temperature in 1× SSC, 0.1% SDS.

Low stringency refers to conditions equivalent to hybridisation in the above described solution at about 50-52° C. In that case, the final wash is performed at the hybridisation temperature in 2× SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al. (1989) ibid or Ausubel, et al. (1995) ibid. Optimal hybridisation conditions have to be determined empirically, as the length and the GC content of the probe also play a role.

B. Immobilisation of Molecular Chaperones/Foldases on Solid Phase Supports

In a preferred aspect, the contact between the thrombin and/or precursor and the molecular chaperone and foldase occurs with the molecular chaperone and/or foldase immobilised on a solid support. Examples of commonly used solid supports include beads, “chips”, resins, matrices, gels, and the material forming the walls of a vessel. Matrices, and in particular gels, such as agarose gels, may conveniently be packed into columns. A particular advantage of solid phase immobilisation is that the reagents may be removed from contact with the polypeptide(s) with facility.

Solid phase materials for use in batch or to be packed into columns are widely available—see for example Sigma's 1999 reagent catalogue entitled “Biochemicals, organic compounds and diagnostic reagents” which includes a range of activated matrices suitable for coupling polypeptides such as cyanogen bromide activated matrices based on sepharose/agarose.

Molecular chaperones/foldases may be immobilised to a solid phase support such as by covalent means or otherwise. A variety of methods for coupling polypeptides to solid phase supports are known in the art. In a preferred aspect of the present invention molecular chaperones and/or preferably foldase polypeptides may be attached to a solid phase support using a method which comprises a reversible thiol blocking step. This is important where the peptide contains a disulphide. An example of such a method is described below.

Preferably, before protection the disulphides are reduced using a reducing agent such as DTT (dithiothreitol), under for example an inert gas, such as argon, to prevent reoxidation. Subsequently, the polypeptide is cyanylated, for example using NTCB (2-nitro, 5-thiocyanobenzoic acid) preferably in stoichiometric amounts, and subjected to controlled hydrolysis at high (non-acidic) pH, for example using NaHCO₃. In the case of DsbA, the pH of the hydrolysis reaction is preferably between 6.5 and 10.5 (the pK of DsbA is 4.0), more preferably between 7.5 and 9.5, and most preferably around about 8.5. The thiols are thus reversibly protected.

The polypeptide is then brought into contact with the solid phase component, for example at between 2.0 and 20.0 mg polypeptide/ml of solid component, preferably between 5.0 and 10.0 and most preferably around about 6.5 mg. The coupling is again carried out at a high (non-acidic) pH, for example using an NaHCO₃ coupling buffer. In the case of DsbA, the pH of the coupling reaction is preferably between 6.5 and 10.5, more preferably between 7.5 and 9.5, and most preferably around about 8.5.

Preferably, after coupling the remaining active groups may be blocked, such as with ethanolamine, and the uncoupled polypeptide removed by washing. Thiol groups may finally be regenerated on the coupled polypeptide by removal of the cyano groups, for example by treatment with DTE or DTT.

C. Methods of Refolding Polypeptides

The present invention provides a method for promoting the correct folding/refolding of a polypeptide selected from thrombin and a precursor thereof which method involves the use of a combination of a molecular chaperone and a foldase. The combination of a molecular chaperone and a foldase provides a synergistic effect on protein folding which results in a greater quantity of active, correctly folded protein being produced than would be expected from a merely additive relationship.

Preferably, one or more of the components used to promote protein folding in accordance with the present invention is immobilised on a solid support. However, both molecular chaperones and foldases may be used in solution. They may be used in free solution, but also in suspension, for example bound to a matrix such as beads, for example sepharose beads, or bound to solid surfaces which are in contact with solutions, such as the inside surfaces of bottles containing solutions, test tubes and the like.

Typically the method of the present invention is used to assist in refolding recombinantly produced thrombin or precursors thereof, which are obtained in an unfolded or misfolded form. Thus, recombinantly produced polypeptides may be contacted with a molecular chaperone and a foldase to unfold, refold and/or reactivate recombinant polypeptides which are inactive due to misfolding and/or are unfolded as a result of their extraction from the host cells in which they were expressed (such as from bacterial inclusion bodies). Such a process may also be termed “reconditioning”.

The method of the invention may be employed to maintain the folded conformation of thrombin and precursors thereof, for example during storage, in order to increase shelf life. Under storage conditions, many proteins lose their activity, as a result of disruption of correct folding. The presence of molecular chaperones, in combination with foldases, reduces or reverses the tendency of polypeptides to become unfolded and thus greatly increases the shelf life thereof.

The method of the invention may be used to promote the correct folding of thrombin and precursors thereof which, through storage, exposure to denaturing conditions or otherwise, have become misfolded. Thus, the invention may be used to recondition thrombin and precursors thereof. For example, thrombin in need of reconditioning may be passed down a column to which is immobilised a combination of a molecular chaperone and a foldase in accordance with the invention. Alternatively, beads having immobilised thereon such a combination may be suspended in a solution comprising the thrombin in need of reconditioning. Moreover, the components of the combination according to the invention may be added in solution to the thrombin in need of reconditioning.

The present invention also provides a method for altering the structure of a polypeptide selected from thrombin and a precursor thereof. Structural alterations include folding, unfolding and refolding. The effect of the alterations is preferably to improve the yield, specific activity and/or quality of the molecule. This may typically be achieved by resolubilising, reconditioning and/or reactivating incorrectly folded molecules post-synthesis.

The terms “reconditioning” and “reactivating” thus encompass in vitro procedures. Particular examples of in vitro procedures may include processing polypeptides that have been solubilised from cell extracts (such as inclusion bodies) using strong denaturants such as urea or guanidium chloride.

The terms “refold”, “reactivate” and “recondition” are not intended as being mutually exclusive. For example, an inactive protein, perhaps denatured using urea, may have an unfolded structure. This inactive protein may then be refolded with a polypeptide of the invention thereby reactivating it. In some circumstances there may be an increase in the specific activity of the refolded/reactivated protein compared to the protein prior to inactivation/denaturation: this is termed “reconditioning”.

The molecule is typically an unfolded or misfolded polypeptide which is in need of folding. Alternatively, however, it may be a folded polypeptide which is to be maintained in a folded state. Preferably, the polypeptide contains at least one disulphide linkage (or two cysteine residues capable of forming such a linkage under suitable conditions).

The invention envisages at least two situations. A first situation is one in which the polypeptide to be folded is in an unfolded or misfolded state, or both. In this case, its correct folding is promoted by the method of the invention. A second situation is one in which the polypeptide is substantially already in its correctly folded state, that is all or most of it is folded correctly or nearly correctly. In this case, the method of the invention serves to maintain the folded state of the polypeptide by affecting the folded/unfolded equilibrium so as to favour the folded state. This prevents loss of activity of an already substantially correctly folded polypeptide. These, and other, eventualities are covered by the reference to “promoting” the folding of the polypeptide.

As used herein, a polypeptide may be unfolded when at least part of it has not yet acquired its correct or desired secondary or tertiary structure. A polypeptide is misfolded when it has acquired at least partially incorrect or undesired secondary or tertiary structure. Techniques are known in the art for assessing polypeptide structure—such as circular dichroism.

Contacting of the thrombin and/or precursor thereof with the chaperone/foldase combination preferably occurs under reducing conditions, such as in the presence of a combination of oxidised glutathione (GSSG) and glutathione (GSH) which act as a redox buffer system and prevent formation of disulphide bonds present in the oxidised state.

A particularly convenient method for contacting the molecular chaperone/foldase combination with the thrombin/precursor involves incubating the thrombin/precursor with the molecular chaperone/foldase combination, whereby the chaperone and foldase are immobilised to sepharose/agarose beads, in a tube, such as an eppendorf tube, in a procedure known as a batch incubation. The tube contents are gently mixed for typically at least 5 minutes, preferably at least 1 to 3 hrs, before allowing the beads to settle by, for example, gravity or low speed centrifugation. The thrombin/precursor in aqueous solution is then simply decanted off.

Another convenient method involves placing a solid phase matrix such as sepharose beads, to which the chaperone and foldase are immobilised, in a chromatography column, applying a sample comprising the polypeptide to be refolded to the top of the column and eluting the polypeptide through the column using a suitable buffer at a suitable rate. Such methods are well known in the art.

The thrombin is preferably bovine thrombin. The term “precursor” means an immature thrombin molecule, such as prethrombin which contains additional polypeptide sequence which are generally removed to form the mature polypeptide. An example of such a precursor is bovine prethrombin-2. Activation to thrombin may be achieved by, for example, incubating the prethrombin with E. carinatus snake venom (see the examples).

The thrombin or precursor to be processed by the method of the invention is typically obtained from cell extracts of host cells expressing recombinant thrombin or its precursor. Host cells include prokaryotes such as E. coli, yeast and insect cells (the baculovirus system is capable of very high level protein expression). Expression of the thrombin or precursor thereof in the host cell is preferably at high levels to maximise yield. However, as discussed above, it is likely that a substantial proportion of the thrombin/precursor will be insoluble and consequently techniques to solubilise normally insoluble components of the cell extracts (such as inclusion bodies) to maximise extraction of the thrombin/precursor will typically be employed. Such techniques include sonication of cells in the presence of strong denaturants such as urea or guanidium chloride.

Solubilised cell extracts may optionally be partially purified by, for example, a variety of affinity chromatography techniques prior to contacting with the chaperone/foldase combination according to the method of the invention.

Thus the starting material for the refolding/reconditioning method of the invention is typically denatured polypeptides in solutions of agents such as urea/guanidium chloride. Alternatively, or in addition, soluble polypeptide samples may be specifically denatured by the addition of appropriate denaturing agents prior to refolding. The untreated thrombin/precursor may be dialysed against a suitable refolding buffer prior to contact with the chaperone/foldase combination if required.

At the end of the refolding/reconditioning process, the refolded thrombin and/or thrombin precursor is typically desalted by dialysis against a suitable storage buffer and/or the use of a desalting column into a suitable storage buffer. Suitable buffers include 25 mM sodium phosphate, 150 mM NaCl and 0.1% PEG 6000 (pH 7.4).

In the case of prethrombin, the polypeptide may then be activated to thrombin by treatment with snake venom (see the examples).

The activity of the refolded/reconditioned thrombin preferably has at least 10% activity relative to wild type thrombin (for example bovine thrombin—available from Sigma), which has been treated in the same way, more preferably at least 12, 13, 14, 15 or 20% activity, or more, such as about 50% activity. Activity may conveniently be assessed using, for example, the chromogenic assay described by Luttenberg et al. 1981 (see the examples).

D. Uses of Refolded/Reconditioned Thrombin/Drecursor Thereof

Thrombin produced by the method of the present invention may be used to cleave polypeptides comprising a thrombin recognition site. In particular, thrombin may be used to aid in the purification of heterologous polypeptides that have been fused to a fusion protein partner such as His×6, GST and the like via a linker comprising a thrombin recognition site.

The invention will now be further described by way of Examples, which are meant to serve to assist one of ordinary skill in the art in carrying out the invention and are not intended in any way to limit the scope of the invention.

### EXAMPLES

Materials and Methods

The overexpression of bovine prethrombin-2 in E. coli was performed as previously described by DiBella et al. (1995). Cells were collected by centrifugation and resuspended in 25 mM Tris, 2 mM EDTA (=ethylenediaminetetraacetic acid), 0.2 mM PMSF (=phenylmethylsulfonyl fluoride), pH 8.0. The cells were cracked using cell disruption and inclusion bodies collected by centrifugation. Inclusion bodies were washed by resuspension in the same buffer and cell disruption followed by centrifugation to recover. Washing was repeated twice with the final resuspension buffer including 1 M urea. Inclusion bodies could be further purified by gel filtration chromatography on a HR10/30 superdex 200 column (Pharmacia). The column was equilibrated in 50 mM sodium phosphate, 8 M urea, 100 mM DTT (=dithiothreitol), pH 7.4. Protein was eluted in the same buffer. The purified pre-enzyme was flash frozen and stored in liquid nitrogen until needed.

Complete unfolding of the prethrombin-2 was achieved by solubilising material in 50 mM sodium phosphate, 8 M urea, 0.3 M DTT, pH 7.4 at a final concentration of ˜2.5 mg/ml. The protein was incubated at 37° C. for 12 hours before dialysing overnight against 100 mM sodium phosphate, 8 M urea, pH 3.0 (adjusted with orthophosphoric acid).

Refolding was initiated by diluting the prethrombin-2 1:100 into 45 ml refolding buffer (50 mM sodium phosphate, 2 mM EDTA, 2 mM GSSG (=oxidised glutathione), 1 mM GSH (=glutathione), 0.3 M L-arginine, pH 7.4) and 5 ml ternary mix resin. The ternary resin contained equal amounts of immobilised DsbA, Cyclophilin A and sht GroEL 191-345 (Altamirano et al., 1999). The refolded prethrombin-2 remained soluble upon removal of urea. Refolding was allowed to proceed at 4° C. for 16 hours with constant end-over-end mixing. The resin was removed by spinning briefly in a bench-top centrifuge and the supernatant recovered. The resin was washed twice in 25 ml refolding buffer including 500 mM sodium chloride and resin removed by spinning briefly in a bench-top centrifuge. Supernatants were combined and dialysed extensively against D-trehalose at 4° C. The volume was reduced to 5 ml using Ultrafree®−15 centrifugal spin concentrators (Millipore) with a 5 KDa molecular weight cut off. The “refolded” protein was then desalted using a Phast™ desalting column (Pharmacia) into 50 mM sodium phosphate, 0.15 M potassium chloride, 2% glycerol, 60 mM guanidine hydrochloride, pH 7.4.

A 1 ml aliquot of desalted prethrombin-2 (˜4.0 μg/ml) was activated to thrombin by adding 10 μl of E. carinatus snake venom (1 mg/ml) and incubating at 37° C. for 2 hours. The snake venom was first pre-treated with p-APMSF and then desalted into 20 mM Tris pH 8.0 buffer using a Phast™ desalting column.

The recovery of active thrombin was assessed by a chromogenic assay (Luttenberg et al., 1981). The chromogenic substrate peptide Bz-Phe-Val-Arg-pNA.HCI (Bachem) at a final concentration of 0.1 mM was added to 800 μl 50 mM sodium phosphate, 0.15 M potassium chloride. 2% glycerol, 60 mM guanidine hydrochloride, pH 7.4 and a 100 μl aliquot of activated thrombin. The absorbance at 405 nm was monitored at room temperature. All absorbance measurements were made on a HP 8453 spectrophotometer. Activity of the refolded thrombin was compared with wild type bovine thrombin (Sigma) that had been treated in a similar manner to the recombinant material.

Results

Recombinant prethrombin-2 was produced unpurified at levels of 40-50 mg/litre of cells as inclusion bodies. The gel filtration purified protein migrated on a 20% SDS Phast™ gel at an apparent mass of 35,000 Da as expected and was ˜50% pure based on Coomassie blue staining.

Chromogenic assay of the recombinant refolded thrombin gave an apparent biological activity of ˜15% compared to the wild type, results are summarised in the following table:

| | | Activity | | |
|---|---|---|---|---|
| | Protein conc. | (absorbance | Activity/ | % wt |
| Protein | in assay | units/time) | protein conc. | activity |
| Wild type | 0.31 μg/ml | 5.79 × 10⁻⁵ | 1.87 × 10⁻⁴ | 100% |
| Recombinant | 0.47 μg/ml | 1.11 × 10⁻⁵ | 2.34 × 10⁻⁵ | 13% |

The recombinant protein had not been subjected to purification procedures (e.g. chromatography on heparin columns.

### REFERENCES

Altamirano, M. M., Garcia, C., Possani, D. A. & Fersht, A. R. (1999) Nature Biotech. 17, 187-191.

DiBella, E. E., Maurer, M. C. & Scheraga, H. A. (1995)J Biol. Chem. 270, 163-169.

Fischer G et al (1984) Biomed Biochim Acta 43:1101-1111.

Luttenberg, R., Christensen, U., Jackson, C. M. & Coleman, P. L. (1981)Methods Enzymol. 80, 341-361

1. A method for promoting the folding of a polypeptide selected from thrombin and a precursor thereof comprising contacting the polypeptide with a molecular chaperone and a foldase.

2. A method according to claim 1 wherein said molecular chaperone and/or foldase are immobilised to a solid phase.

3. A method according to claim 2 wherein the solid phase is a matrix.

4. A method according to claim 3 wherein the matrix is present in a chromatography column.

5. A method according to claim 1 wherein the polypeptide is an unfolded or misfolded polypeptide.

6. A method according to claim 1 wherein the molecular chaperone is an hsp-60 chaperonin or fragment thereof having refolding activity.

7. A method according to claim 6, wherein the molecular chaperone fragment comprises a region consisting of fragments 191-376, 191-345, 191-335 or 193-335 of the sequence of E. Coli groel or a homologue thereof.

8. A method according to claim 1, wherein the foldase is selected from a thiol/disulphide oxidoreductase and a peptidyl-prolyl isomerase.

9. A method according to claim 8, wherein the thiol/disulphide oxidoreductase is selected from E. coli DsbA and mammalian protein disulphide isomerase.

10. A method according to claim 8, wherein the peptidyl prolyl isomerase is selected from cyclophilin, parbulen, SurA and FK506 binding proteins.

11. A method according to claim 1 comprising contacting the polypeptide with a molecular chaperone and both a thiol/disulphide oxidoreductase and a peptidyl-prolyl isomerase.

12. A method according to claim 11 wherein the thiol/disulphide oxidoreductase is DsbA and the peptidyl-prolyl isomerase is cyclophilin A.

13. A method according to claim 1 wherein the contacting 10 takes place under reducing conditions.

14. A method according to claim 1 wherein the polypeptide has been expressed in a host cell selected from a prokaryote, a yeast and an insect cell.

15. Use of a molecular chaperone and one or more foldases for promoting the folding of a polypeptide selected from thrombin and a precursor thereof.

16. A polypeptide selected from thrombin and a precursor thereof obtainable by the method of claim 1.

17. Use of a polypeptide according to claim 16 in protein purification.

18. Use of a polypeptide according to claim 16 in cleaving a heterologous polypeptide.

19. Use according to claim 18 wherein the heterologous polypeptide has been produced recombinantly.
